(19) United States
(12) Patent Application Publication
(10) Pub. No.: US 2025/0125055 A1
(43) Pub. Date: Apr. 17, 2025

(54) METHODS AND SYSTEMS FOR JOINT MINIMIZATION OF MULTI-ORGAN SYSTEM RISKS FOR TIME-VARYING TREATMENT OPTIMIZATION

(71) Applicant: KONINKLIJKE PHILIPS N.V., EINDHOVEN (NL)

(72) Inventors: YALE CHANG, BEDFORD, MA (US); TAKAHIRO KIRITOSHI, CAMBRIDGE, MA (US)

(21) Appl. No.: 18/682,005

(22) PCT Filed: Jul. 22, 2022
(86) PCT No.: PCT/EP2022/070665

§ 371 Date: Feb. 7, 2024

Related U.S. Application Data
(60) Provisional application No. 63/232,244, filed on Aug. 12, 2021.

(51) Int. Cl. G16H 50/30; G16H 20/10; G16H 50/70

(52) U.S. Cl. CPC G16H 50/30; G16H 20/10; G16H 50/70

(57) ABSTRACT

A method (100) for recommending a patient treatment comprising: (i) receiving (120) information about the patient, wherein the information comprises a plurality of patient outcome prediction features; (ii) extracting (130) the plurality of patient outcome prediction features from the received information; (iii) analyzing (140), using a trained time-varying treatment effect model, the plurality of patient outcome prediction features to predict a plurality of different outcomes for the patient, wherein each of the plurality of different outcomes is associated with a patient treatment leading to said respective outcome; (iv) identifying (150) at least one of the plurality of different outcomes and treatment as a recommended outcome and associated treatment for the patient, wherein identifying comprises identification of an outcome and associated treatment that maximizes favorable results for two or more organ systems for the patient; and (v) providing (160) the recommended outcome and associated treatment for the patient to a user.

### FIELD OF THE DISCLOSURE

The present disclosure is directed generally to methods and systems for recommending a patient treatment, using a patient outcome prediction system, that optimizes favorable outcomes for two or more organ systems.

### BACKGROUND

Time-varying treatment optimization aims to dynamically choose the optimal treatment leading to the best treatment outcome for a patient. Existing time-varying treatment effect estimation approaches only optimize a single treatment outcome, such as blood pressure or lactate. However, the aggressive optimization of the outcome of one organ system could potentially increase the risk of other organ systems. For example, for sepsis patients, a vasopressor is used to increase a patient's blood pressure and therefore stabilize the hemodynamic condition. However, the administration of large amounts of vasopressors can increase the burden of the kidney and risk of renal failure. As another example, for patients suffering from both hemodynamic instability and acute respiratory distress syndrome (ARDS), the administration of fluids can increase blood pressure and stabilize the hemodynamic condition. However, large amounts of fluids can cause severe edema and therefore significantly increase the patient's risk of respiratory failure.

### SUMMARY OF THE DISCLOSURE

Accordingly, there is a continued need for methods and systems that generate and provide a patient treatment that optimizes favorable outcomes for two or more organ systems. Various embodiments and implementations herein are directed to a method and system configured to generate and present a recommended treatment and outcome for a patient using a patient outcome prediction system. The system receives information about the patient, comprising a plurality of patient outcome prediction features. The system then extracts the plurality of patient outcome prediction features from the received information, and analyzes these features with a trained time-varying treatment effect algorithm to predict a plurality of different outcomes for the patient. Each of the plurality of different outcomes is associated with a patient treatment leading to said respective outcome. The system identifies one of the outcomes, and the associated treatment, as a recommended treatment and outcome for the patient, wherein an outcome is identified as recommended when it maximizes favorable results for two or more organ systems for the patient. The system provides the recommended treatment and outcome for the patient to a user via a user interface of the patient outcome prediction system.

Generally, in one aspect, a method for recommending a patient treatment using a patient outcome prediction system is provided. The method includes: (i) receiving, at the patient outcome prediction system, information about the patient, wherein the information comprises a plurality of patient outcome prediction features; (ii) extracting, by a processor of the patient outcome prediction system, the plurality of patient outcome prediction features from the received information; (iii) analyzing, using a trained time-varying treatment effect algorithm of the patient outcome prediction system, the plurality of patient outcome prediction features to predict a plurality of different outcomes for the patient, wherein each of the plurality of different outcomes is associated with a patient treatment leading to said respective outcome; (iv) identifying at least one of the plurality of different outcomes and the treatment associated with the selected outcome as a recommended outcome and associated treatment for the patient, wherein identifying comprises identification of an outcome and associated treatment that maximizes favorable results for two or more organ systems for the patient; and (v) providing, to a user via a user interface of the patient outcome prediction system, the recommended outcome and associated treatment for the patient.

According to an embodiment, providing comprises providing two or more of the plurality of different outcomes treatments as possible or recommended outcomes and associated treatments for the patient.

According to an embodiment, the method further includes the step of receiving, via the user interface, a selection of one of the two or more of the plurality of different outcomes treatments provided via the user interface.

According to an embodiment, the method further includes the step of implementing the provided recommended treatment for the patient.

According to an embodiment, identifying at least one of the plurality of different outcomes and the treatment associated with the selected outcome as a recommended outcome and associated treatment for the patient comprises the steps of: comparing the predicted plurality of different outcomes to each other; and selecting one of the plurality of different outcomes as an outcome that maximizes favorable results for two or more organ systems for the patient, if no other outcome has a more favorable outcome.

According to an embodiment, the identifying step comprises a Pareto-optimal analysis.

According to an embodiment, the trained time-varying treatment effect algorithm comprises a G-formula approach.

According to an embodiment, the method further includes the step of training the time-varying treatment effect algorithm of the patient outcome prediction system using historical patient data.

According to a second aspect is a system for recommending a patient treatment. The system includes: a trained time-varying treatment effect model configured to predict a plurality of different outcomes for a patient using a plurality of patient outcome prediction features for the patient; a processor configured to: (i) receiving information about the patient, wherein the information comprises the plurality of patient outcome prediction features; (ii) extract the plurality of patient outcome prediction features from the received information; (iii) analyze, using the trained time-varying treatment effect model, the plurality of patient outcome prediction features to predict a plurality of different outcomes for the patient, wherein each of the plurality of different outcomes is associated with a patient treatment leading to said respective outcome; (iv) identify at least one of the plurality of different outcomes and the treatment associated with the selected outcome as a recommended outcome and associated treatment for the patient, wherein identifying comprises identification of an outcome and associated treatment that maximizes favorable results for two or more organ systems for the patient; and a user interface configured to provide the recommended outcome and associated treatment for the patient to a user.

According to an embodiment, the processor is further configured to train the time-varying treatment effect model using historical patient data.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for recommending a patient treatment, in accordance with an embodiment.

FIG. 2 is a schematic representation of a patient outcome prediction system, in accordance with an embodiment.

FIG. 3 is a flowchart of a method for training a patient outcome prediction system, in accordance with an embodiment.

FIG. 4 is a schematic representation of a time-varying treatment effect estimation, in accordance with an embodiment.

FIG. 5 is a schematic representation of the G-formula for time-varying treatment effect estimation, in accordance with an embodiment.

### DETAILED DESCRIPTION OF EMBODIMENTS

The present disclosure describes various embodiments of a system and method configured to generate and present a patient treatment that optimizes favorable outcomes for two or more organ systems. More generally, Applicant has recognized and appreciated that it would be beneficial to provide a method and system to provide an optimal patient treatment. Accordingly, a trained patient outcome prediction system predicts an optimal treatment/outcome recommendation and communicates the recommendation to a clinician, which allows the clinician to make more informed treatment decisions. The trained patient outcome prediction system receives information about a patient, the information including a plurality of patient outcome prediction features. These features are extracted from the received information and analyzed by a trained time-varying treatment effect algorithm of the system to predict a plurality of different outcomes for the patient, where each of the plurality of different outcomes is associated with a patient treatment leading to said respective outcome. The system identifies one of the outcomes, and the associated treatment, as a recommended treatment and outcome for the patient, wherein an outcome is identified as recommended when it maximizes favorable results for two or more organ systems for the patient. The system provides the recommended treatment and outcome for the patient to a user via a user interface of the patient outcome prediction system.

According to an embodiment, the systems and methods described or otherwise envisioned herein can, in some non-limiting embodiments, be implemented as an improvement to existing commercial products for patient analysis or monitoring, such as an Intellivue Guardian bedside monitor, or a Central Station (both available from Koninklijke Philips NV, the Netherlands), or a clinical decision support system, or any other suitable patient or care facility system.

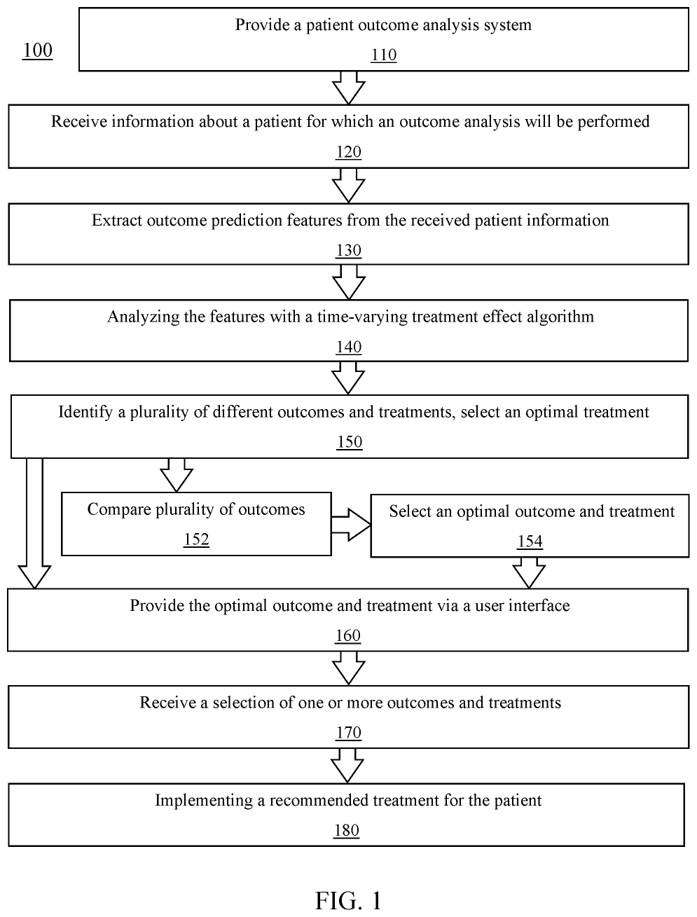

Referring to FIG. 1, in one embodiment, is a flowchart of a method 100 for recommending a patient treatment using a patient outcome prediction system. The methods described in connection with the figures are provided as examples only, and shall be understood not limit the scope of the disclosure. The patient outcome prediction system can be any of the systems described or otherwise envisioned herein. The patient outcome prediction system can be a single system or multiple different systems.

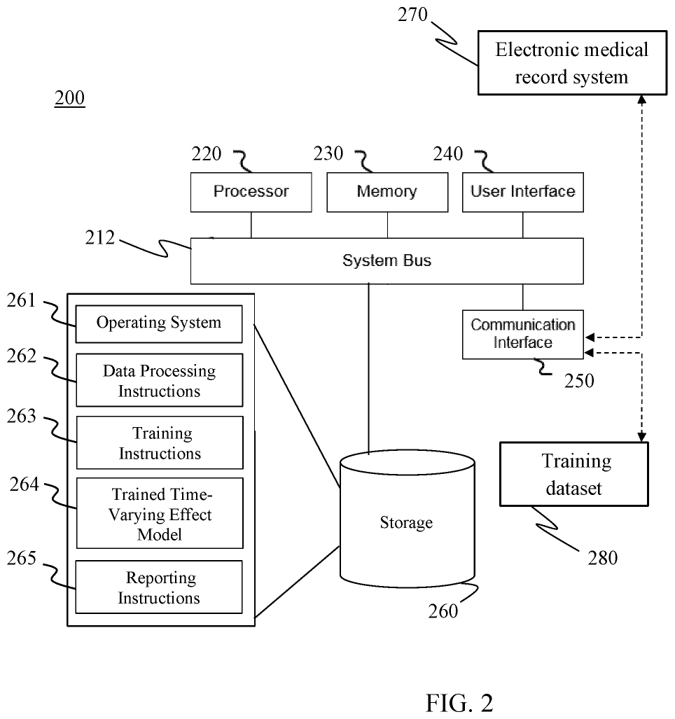

At step 110 of the method, a patient outcome prediction system is provided. Referring to an embodiment of a patient outcome prediction system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, patient outcome prediction system 200 can be any of the systems described or otherwise envisioned herein. Other elements and components of patient outcome prediction system 200 are disclosed and/or envisioned elsewhere herein.

At step 120 of the method, the patient outcome prediction system receives information about a patient for which an analysis will be performed. According to an embodiment, the patient information comprises a plurality of features about the patient. Only some, or all, of the information about the patient may ultimately be utilized by the patient outcome prediction system. The plurality of features may comprise, for example, vital sign information about the patient, including but not limited to physiologic vital signs such as heart rate, blood pressure, respiratory rate, apnea, SpO₂, invasive arterial pressure, noninvasive blood pressure, and more. According to an embodiment, the information may also comprise medical information about the patient, including but not limited to demographics, physiological measurements other than vital data such as physical observations, and/or patient diagnosis, among many other types of medical information. As an example, the medical information can include detailed information on patient demographics such as age, gender, and more; diagnosis or medication condition such as cardiac disease, psychological disorders, chronic obstructive pulmonary disease, and more. Many other types of medical information are possible. Accordingly, the received information can be any information relevant to a patient outcome prediction.

The patient outcome prediction system can receive patient information from a variety of different sources, including any source that comprises one or more patient features. According to an embodiment, the patient outcome prediction system is in communication with an electronic medical records database from which the patient information and one or more of the plurality of features may be obtained or received. The electronic medical records database may be a local or remote database and is in communication with the patient outcome prediction system 200. According to an embodiment, the patient outcome prediction system comprises an electronic medical record database or system 270 which is optionally in direct and/or indirect communication with system 200. According to another embodiment, the patient outcome prediction system may obtain or receive the plurality of features from equipment or a healthcare professional obtaining that information directly from the patient. According to an embodiment, the patient outcome prediction system may query an electronic medical record database or system, comprising fast healthcare interoperability resources (FHIR) for example, to obtain the patient information.

The patient information received by the patient outcome prediction system may be processed by the system according to methods for data handling and processing/preparation, including but not limited to the methods described or otherwise envisioned herein. The patient information received by the system may be utilized, before or after processing, immediately or may be stored in local or remote storage for use in further steps of the method.

At step 130 of the method, the patient outcome prediction system extracts a plurality of patient outcome prediction features from the patient information received in step 120 of the method. The plurality of patient outcome prediction features may comprise a predetermined list of patient features. The system is trained to identify and extract the patient features from the received patient information using any of a wide variety of algorithms, methods, or systems for identifying and extracting patient data. The plurality of identified and extracted patient outcome prediction features may be utilized immediately or may be stored in local or remote storage for use in further steps of the method.

At step 140 of the method, the patient outcome prediction system analyzes the extracted plurality of patient outcome prediction features using a trained time-varying treatment effect algorithm of the patient outcome prediction system to predict a plurality of different outcomes for the patient. Each of the plurality of different outcomes is associated with a patient treatment leading to said respective outcome. The trained model can be any time-varying treatment effect algorithm, module, or component configured to analyze input data to classify the input or otherwise generate or predict a plurality of different outcomes. Aspects and embodiments of the trained time-varying treatment effect algorithm of the patient outcome prediction system are further described or envisioned herein.

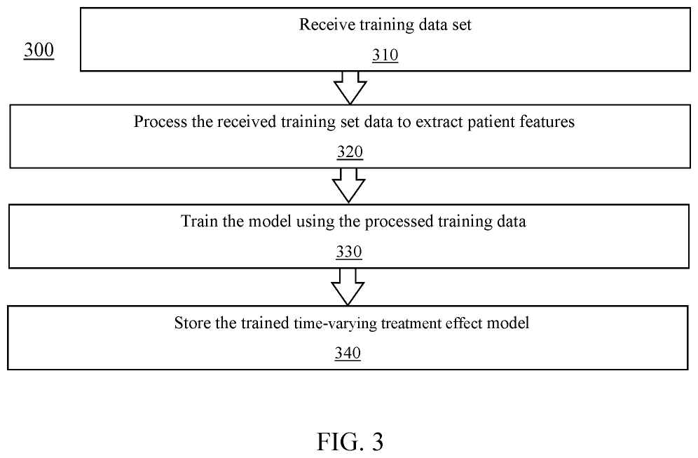

Referring to FIG. 3, in one embodiment, is a method 300 for training the time-varying treatment effect algorithm of the patient outcome prediction system. At step 310 of the method, the system receives a training data set comprising training data about a plurality of patients. The training data can comprise any patient information suitable for training a time-varying treatment effect algorithm. For example, the patient information may comprise information such as patient conditions, patient treatment, patient diagnosis, healthcare facility visits or admissions, age, gender, height, weight, comorbidities, dietary information, and other information. The patient information may further comprise medical information such as vital sign information about the patient, including but not limited to physiologic vital signs, physiological measurements other than vital data such as physical observations, patient diagnosis or medication condition, and more, among many other types of information. The training data may be stored in and/or received from one or more databases. The database may be a local and/or remote database. For example, the system may comprise a database of training data, such as database 280 in FIG. 2.

According to an embodiment, the patient outcome prediction system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract patient outcome prediction features about one or more of the plurality of patients. The patient outcome prediction features may be any features which will be utilized to train the time-varying treatment effect algorithm, such as any patient outcome prediction features that can or will be utilized by the trained time-varying treatment effect algorithm for outcome prediction for a future patient. Feature extraction can be accomplished by a variety of embodiments for feature identification, extraction, and/or processing, including any method for extracting features from a dataset. The outcome of a feature processing step or module of the patient outcome prediction system is a set of patient outcome prediction features about a plurality of patients, which thus comprises a training data set that can be utilized to train the classifier.

At step 330 of the method, the system trains the time-varying treatment effect algorithm, which will be the algorithm utilized in analyzing input information as described or otherwise envisioned. The time-varying treatment effect algorithm is trained using the extracted features according to known methods for training a machine learning algorithm. According to an embodiment, the algorithm is trained, using the processed training dataset, to generate a outcome and treatment recommendation for a patient. At step 340 of the method, the trained model is stored for future use. According to an embodiment, the model may be stored in local or remote storage.

At step 150 of the method, according to an embodiment, the system identifies, among the plurality of predicted different outcomes and the treatment associated with the selected outcome, at least one outcome and the associated treatment as a recommended treatment and outcome for the patient. According to an embodiment, the system identifies an outcome and its associated treatment as a recommended treatment and outcome for the patient if that outcome maximizes favorable results for two or more organ systems for the patient, rather than for just a single organ system. Possible processes for identifying an outcome as maximizing favorable results for two or more organ systems for the patient are described or otherwise envisioned herein.

According to an embodiment, identifying an outcome as maximizing favorable results for two or more organ systems for the patient comprises a multi-objective optimization, also known as a Pareto-optimal analysis. The multi-objective optimization or analysis identifies an optimal decision when there are two or more possibly conflicting objectives. For example, an optimal decision for a first organ system may not be optimal for a second organ system, and vice versa. However, there may exist a decision—here, a treatment option—that simultaneously optimizes outcomes for both the first and second organ systems. A treatment (i.e., “solution”) that optimizes both organ systems, with no better possible or predicted outcome for both organ systems simultaneously, is a dominant or nondominated solution. Thus, a solution is a Pareto optimal or nondominated solution if none of the outcomes can be improved in value without degrading some of the other outcomes.

Accordingly, at optional step 152 of the method, the system compares the predicted plurality of different outcomes to each other. This may comprise, for example, comparison of the outcome of each of the multiple organ systems in a first predicted outcome to the outcome of each of the multiple organ systems in a second predicted outcome, and so on. Many other methods and approaches for comparison are possible. Thus, at optional step 154 of the method, the system selects, based on the comparison at step 152, one of the plurality of different outcomes as an outcome that maximizes favorable results for two or more organ systems for the patient, if no other outcome has a more favorable outcome.

At step 160 of the method, the recommended treatment and outcome for the patient is provided to a user via a user interface of the patient outcome prediction system. The display may also comprise information about the patient, the input data for the patient, and/or one or more additional recommended treatments and outcomes for the patient, including those that were not selected because they were not optimal. Other information is possible. The report may be communicated by wired and/or wireless communication to another device. For example, the system may communicate the report to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands.

At optional step 170 of the method, the system may receive, from the user interface, a selection of one or more recommended treatments and outcomes for the patient. For example, the system may present multiple recommended treatments and outcomes for the patient that are generated by the system. The presentation may include a ranked list of recommended treatments and outcomes for the patient. These possible recommended treatments and outcomes for the patient may comprise a score or other quantified indication of the outcome, and this score or other quantified indication may be utilized to rank the possible recommended treatments and outcomes for the patient. The system may present this to the user in a way that allows the user to select one of the presented plurality of recommended treatments and outcomes for the patient. For example, the user can click a displayed recommended treatment and outcome, provide an audible selection of a displayed recommended treatment and outcome, or utilize any other method for selection via any user interface.

At step 180 of the method, the clinician or other decisionmaker utilizes the displayed and/or selected recommended outcome and associated treatment in patient care decision-making. For example, the clinician or other decisionmaker can implement the displayed and/or selected treatment. This implementation can be an attempt by the clinician to effect the outcome, selected as optimal, with which the outcome is associated, which will maximize the best outcomes for the two or more organ systems. Implementing the displayed and/or selected treatment can comprise a wide variety of actions, including administering, adjusting, or removing a medication, ordering a test, and many other possible care actions.

Referring to FIG. 2 is a schematic representation of a patient outcome prediction system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, trained time-varying effect model 264, and/or reporting instructions 265.

According to an embodiment, the electronic medical record system 270 is an electronic medical records database from which the information about the patient, including the plurality of outcome prediction features, may be obtained or received. The electronic medical records database may be a local or remote database and is in communication the patient risk score analysis system 200. According to an embodiment, the patient outcome prediction system comprises an electronic medical record database or system 270 which is optionally in direct and/or indirect communication with system 200.

According to an embodiment, the training data set 280 is a dataset that may be stored in a local or remote database and is in communication the patient outcome prediction system 200. According to an embodiment, the patient outcome prediction system comprises a training data set 280. The training data can comprise medical information about a patient, including but not limited to demographics, physiological measurements such as vital data, physical observations, and/or diagnosis, and treatments among many other types of medical information.

According to an embodiment, embodiment, data processing instructions 262 direct the system to retrieve and process input data which is used to train the time-varying effect model 264. The data processing instructions 262 direct the system to for example, receive or retrieve input data or medical data to be used by the system as needed, such as from electronic medical record system 270 among many other possible sources. As described above, the input data can comprise a wide variety of input types from a wide variety of sources.

According to an embodiment, the data processing instructions 262 also direct the system to process the input data to generate a plurality of patient outcome prediction features related to medical information, outcomes, and treatments for a plurality of patients, which are used to train the classifier. This can be accomplished by a variety of embodiments for feature identification, extraction, and/or processing. The outcome of the feature processing is a set of outcome prediction features related to patient outcome risk analysis for a patient, which thus comprises a training data set that can be utilized to train the risk model 264.

According to an embodiment, training instructions 263 direct the system to utilize the processed data to train the time-varying effect model 264. The time-varying effect model can be any machine learning algorithm, classifier, or model sufficient to utilize the type of input data provided, and to generate a time-varying effect analysis. Thus, the system comprises a trained time-varying effect model 264 to generate a plurality of outcomes and associated treatments, as described or otherwise envisioned herein.

According to an embodiment, reporting instructions 265 direct the patient outcome prediction system to generate and provide a report to a user via user interface 240 comprising a generated recommended treatment and outcome for the patient. The display and information may also comprise information about the patient, the input data for the patient, and/or one or more additional recommended treatments and outcomes for the patient, including those that were not selected because they were not optimal. Other information is possible. The report may be communicated by wired and/or wireless communication to another device. For example, the system may communicate the report to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands.

According to an embodiment of the system, the system determines an optimal treatment leading to the best clinical outcome for the patient using provided demographics, vital signs, clinical measurements and/or interventions for the patient. The system jointly minimizes the risk of multiple organ systems for time-varying treatment effect estimation. The learned therapy decision support model can avoid the recommendation of overly aggressive treatment strategy that leads to improvement of one organ system while failure of other organ systems. For example, for a sepsis patient, the treatment decision could be different dosages of norepinephrine. The clinical outcome could include: 1) lactate clearance; 2) elevation of blood pressure; and 3) long-term survival, as just one example. The system can therefore provide therapy decision support to clinicians. A possible use case is as follows:
1. A user (such as a clinician) enters a query patient with associated information such as demographics, comorbidities, vitals, labs, and/or other information;
2. A patient explorer returns a set of similar patients with the query patient in terms of user-specified filter criteria or using the similarity metric learned by the machine learning model;
3. The similar patients comprise different patterns of treatment options and clinical outcomes; and
4. The time-varying treatment effect estimation system and method described or otherwise envisioned herein is applied to estimate or predict potential outcomes of different treatments, and therefore can provide therapy decision support to clinicians.

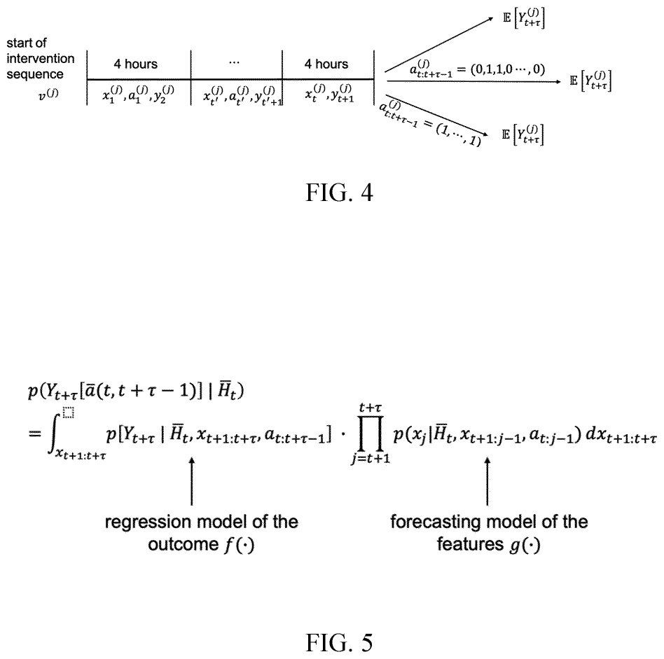

Referring to FIG. 4, in one embodiment, is a schematic representation of a time-varying treatment effect estimation. FIG. 4 shows the problem definition of time-varying treatment effect estimation, where:
The patient stay in the ICU is divided into multiple 4-hour segments (although many other time periods or segments are possible);
xₜ^{(j)} represents the dynamic feature vector (such as vitals and lab results, although many other types of information are possible) measured at the start of the t-th segment of the j-th patient;
v^{(j)} represents the static feature vector (such as demographics and admission diagnosis, among other information) of the j-th patient;
aₜ^{(j)} represents the treatment applied to the j-th patient during the t-th segment; and
yₜ₊₁^{(j)} represents the outcome observed after the end of the t-th segment (assumed to be observed at the start of the (t+1)-th segment) for the j-th patient.

According to an embodiment, an objective of time-varying treatment effect estimation is to estimate the potential treatment outcome [symbol] [Y_{t+τ}^{(j)}], which is the expected outcome observed at the start of the (t+τ)-th segment, under different potential treatment sequences applied to the patient from the t-th to the (t+τ−1)-th segment (denoted as at a_{t:t+τ-1}^{(j)}). The treatment sequence leading to the optimal treatment outcome [symbol] [Y_{t+τ}^{(j)}] can be recommended to the user, such as a clinician or other decisionmaker.

[symbol]

Since long-term forecasting is a fundamentally difficult problem, in practice, instead of comparing multiple treatment sequences with lengths greater than 1, τ=1 can be set and one-step-ahead prediction of treatment outcomes can be applied under different treatment options.

In the epidemiology community, the treatment outcome is often selected as patient survival or mortality. According to an embodiment, for application of treatment optimization for sepsis patients, besides mortality, other short-term clinical outcomes, including lactate, blood pressure can be used.

The time-varying treatment effect estimation is similar to reinforcement learning (RL) because both approaches are built on sequence decision process. However, there are a few important distinctions between these two approaches, which are listed in Table 1. Specifically, the treatment strategy learned from the potential outcome model seems more interpretable compared to the policy function in RL, e.g. it can learn the following statement: use vasopressors rather than fluids because vasopressors can lead to greater increase of blood pressure. In contrast, the learned RL policy is based on the reward function, which is often difficult to interpret after combining multiple clinical outcomes.

TABLE 1
Differences between time-varying treatment effect estimation and
reinforcement learning.

| | Potential Treatment Outcomes | |
|---|---|---|
| | Under Time-Varying Treatments | Reinforcement Learning |
| Model | potential treatment outcome | policy function: a ← π(s) |
| output | estimation [symbol] (Y_{t+τ}[ā(t, t + τ − | |
| | 1)] \| H̅ₜ) | |
| Markovian | No: xₜ does not summarize the | Yes: sₜ does not depend on |
| assumption? | history | s_{t-M} (M ≥ 2) |
| Reward | No: no reward function needs | Yes: trial and error of the |
| Engineering? | to be defined | reward design |

Motivated by these clinical observations, the systems and methods described or otherwise envisioned herein choose the treatment that can jointly minimize the risks of multiple organ systems rather than a single organ system. As just one example, these organ systems can include 1) the hemodynamic system; 2) the renal system; and/or 3) the respiratory system. Examples of clinical outcomes characterizing each organ system are listed in Table 2, although these examples are non-limiting.

TABLE 2
Clinical outcomes of possible organ systems

| Organ System | Clinical outcomes |
|---|---|
| Hemodynamics | Blood pressure, lactate |
| Renal | Creatinine, urine output, AKI |
| Respiratory | PF ratio, ventilation status |

The optimization of multiple clinical outcomes can be solved by the Pareto or multi-objective optimization defined as shown in Eq. 1:

$${min}_{a} ( {r}_{hemodynamics} ( a ) , {r}_{renal} ( a ) , {r}_{respiratory} ( a ) ) \qquad ( Eq . 1 )$$

Where:
r_{hemodynamics}(a) represent the risk of the hemodynamic system induced by the treatment a. If blood pressure is used as the markers of the hemodynamic condition, the system can define the risk function r_{hemodynamics}(a) to encourage the exit from the severe hypotension state, i.e. low blood pressure would correspond to high hemodynamic risk;

For rᵣₑₙₐₗ (a), higher level of creatinine would correspond to higher risk of renal failure; and

For r_{respiratory}(a), following the Berlin definition, lower value of PF ratio would correspond to higher risk of respiratory failure.

Note that this formulation is different from the regular training scheme that minimizes the weighted linear combination of these individual risk functions, as shown in Eq. 2:

$${min}_{a} {λ}_{1} \cdot {r}_{hemodynamics} ( a ) + {λ}_{2} \cdot {r}_{renal} ( a ) + {λ}_{3} \cdot {r}_{respiratory} ( a ) \qquad ( Eq . 2 )$$

Minimizing the weighted linear combination of individual risk functions is only valid if these risks are not competing with each other. However, as shown in the example of vasopressors for sepsis patient treatment, the risks of the hemodynamic system and that of the renal system are competing. The Pareto optimization can identify a better solution that minimizes the risks of multiple organ systems simultaneously through searching for the Pareto-stationary point.

According to an embodiment, the systems and methods described or otherwise envisioned herein: (1) estimate the expected outcomes of multiple organ systems through time-varying treatment effect estimation; and (2) choose an optimal treatment strategy through discrete Pareto-optimization.

According to an embodiment, the estimation of expected outcomes through time-varying treatment effect estimation can be achieved multiple different ways. As non-limiting examples are the G-formula, inverse propensity weighted marginal structure model (IPW-MSM), and the G-estimation of structural nested models. Provided herein is a summary of the general idea and advantages/disadvantages of the G-formula and IPW-MSM approaches. Although these two approaches are discussed in detail, it shall be understood that these examples are non-limiting and that other approaches for time-varying treatment effect estimation can be utilized.

According to an embodiment, the IPW-MSM approach generates a pseudo-population in which treatments are independent of confounders, enabling the estimation of the parameters of the marginal structural model. The idea is based on importance sampling, and the approach has the advantage of low bias but could have high variance.

According to an embodiment, the G-formula approach models the joint density of observed data to generate potential outcomes under different treatment options, and is based on direct modelling between the input variables and the outcome (i.e., parametric regression models). The G-formula approach has the advantage of low variance. However, it would be biased if the model is mis-specified.

Referring to FIG. 5, in one embodiment, is the G-formula, including the regression model f(⋅) and the forecasting model g(⋅). In the training phase, the regression model f(⋅) and the forecasting model g(⋅) are learned. Both traditional methods based on generalized linear models and the more recent approaches bases on the recurrent neural networks (RNNs) can be applied to learn f(⋅) and g(⋅).

According to an embodiment, in the testing phase, the treatment sequence a_{t:t+τ-1} is given and fixed:
1. Sample multiple trajectories of features x_{t+1:t+τ} using the forecasting model g(⋅);
2. For each trajectory, apply the outcome regression model f(⋅); and
3. Take the Monte-Carlo average as the estimate of the expectation.

According to an embodiment, the system can build a G-formula model to estimate the potential outcome of each organ system. Therefore, multiple models can be needed to estimate the outcomes of multiple organ systems.

According to an embodiment, for Pareto-optimization a feasible solution a₁ is said to dominate another solution a₂ if the following two conditions are satisfied: (i) rᵢ(a₁)≤rᵢ(a₂) holds for all indices i, where each i corresponds to one organ system; and (2) rᵢ(a₁)<rᵢ(a₂) holds for at least one index i. A solution a* is called Pareto optimal if there does not exist another solution that dominates it. According to an embodiment, the objection of the systems and methods described or otherwise envisioned herein is to identify a Pareto-optimal solution that jointly minimize the risks of multiple organ systems.

If the treatment variable a is continuous, the Pareto-optimal solution can be reached through the multiple-gradient descent algorithm (MGDA). On the other hand, if the treatment variable a is discrete, augmented weighted Tchebychev scalarizations can be applied. However, in the case of 1) discrete treatment variable, and 2) one-step-ahead prediction, the discrete values a can take is often no more than five. It would make sense to simply make decision based on the look-up table shown in Table 3.

TABLE 3
Look-up table used to determine Pareto-optimal treatment level a.

| Treatment | r_{hemodynamics} | | |
|---|---|---|---|
| level a | (a) | rᵣₑₙₐₗ (a) | r_{respiratory} (a) |
| 1 | | | |
| 2 | | | |
| 3 | | | |
| 4 | | | |
| 5 | | | |

Following the definition of Pareto-optimal solution, each treatment level can be compared against other levels check whether it is dominated by any other solution. That level would be Pareto-optimal if there does not exist any other level that dominate it. Otherwise, it will not be Pareto-optimal.

According to an embodiment, the patient outcome prediction system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the patient outcome prediction system. Thus, generating a functional and skilled trained classifier comprises a process with a volume of calculation and analysis that a human brain cannot accomplish in a lifetime, or multiple lifetimes.

In addition, the patient outcome prediction system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime.

By providing recommended patient outcomes and associated treatments, this novel patient outcome prediction system has an enormous positive effect on patient treatment and outcomes, especially when weighing outcomes of two or more organ systems. As just one example in a clinical setting, by providing a system that can provide recommended outcomes and associated treatments, the system can facilitate treatment decisions and improve survival outcomes, thereby leading to saved lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for recommending a patient treatment using a patient outcome prediction system, comprising:
receiving, at the patient outcome prediction system, information about the patient, wherein the information comprises a plurality of patient outcome prediction features;
extracting, by a processor of the patient outcome prediction system, the plurality of patient outcome prediction features from the received information;
analyzing, using a trained time-varying treatment effect algorithm of the patient outcome prediction system, the plurality of patient outcome prediction features to predict a plurality of different outcomes for the patient, wherein each of the plurality of different outcomes is associated with a patient treatment leading to said respective outcome;
identifying at least one of the plurality of different outcomes and the treatment associated with the selected outcome as a recommended outcome and associated treatment for the patient, wherein identifying comprises identification of an outcome and associated treatment that maximizes favorable results for two or more organ systems for the patient; and
providing, to a user via a user interface of the patient outcome prediction system, the recommended outcome and associated treatment for the patient.

2. The method of claim 1, wherein providing comprises providing two or more of the plurality of different outcomes treatments as possible or recommended outcomes and associated treatments for the patient.

3. The method of claim 2, further comprising the step of receiving, via the user interface, a selection of one of the two or more of the plurality of different outcomes treatments provided via the user interface.

4. The method of claim 1, further comprising the step of implementing the provided recommended treatment for the patient.

5. The method of claim 1, wherein identifying at least one of the plurality of different outcomes and the treatment associated with the selected outcome as a recommended outcome and associated treatment for the patient comprises the steps of:
comparing the predicted plurality of different outcomes to each other; and
selecting one of the plurality of different outcomes as an outcome that maximizes favorable results for two or more organ systems for the patient, if no other outcome has a more favorable outcome.

6. The method of claim 4, wherein said identifying step comprises a Pareto-optimal analysis.

7. The method of claim 1, wherein said trained time-varying treatment effect algorithm comprises a G-formula approach.

8. The method of claim 1, further comprising the step of training the time-varying treatment effect algorithm of the patient outcome prediction system using historical patient data.

9. A system for recommending a patient treatment, comprising:
a trained time-varying treatment effect model configured to predict a plurality of different outcomes for a patient using a plurality of patient outcome prediction features for the patient;
a processor configured to: receiving information about the patient, wherein the information comprises the plurality of patient outcome prediction features; extract the plurality of patient outcome prediction features from the received information; analyze, using the trained time-varying treatment effect model, the plurality of patient outcome prediction features to predict a plurality of different outcomes for the patient, wherein each of the plurality of different outcomes is associated with a patient treatment leading to said respective outcome; identify at least one of the plurality of different outcomes and the treatment associated with the selected outcome as a recommended outcome and associated treatment for the patient, wherein identifying comprises identification of an outcome and associated treatment that maximizes favorable results for two or more organ systems for the patient; and
a user interface configured to provide the recommended outcome and associated treatment for the patient to a user.

10. The system of claim 9, wherein the user interface is configured to provide two or more of the plurality of different outcomes treatments as possible or recommended outcomes and associated treatments for the patient.

11. The system of claim 10, wherein the system is further configured to receive, via the user interface, a selection of one of the two or more of the plurality of different outcomes treatments provided via the user interface.

12. The system of claim 10, wherein identifying at least one of the plurality of different outcomes and the treatment associated with the selected outcome as a recommended outcome and associated treatment for the patient comprises the steps of:
comparing the predicted plurality of different outcomes to each other; and
selecting one of the plurality of different outcomes as an outcome that maximizes favorable results for two or more organ systems for the patient, if no other outcome has a more favorable outcome.

13. The system of claim 12, wherein said identifying comprises a Pareto-optimal analysis.

14. The system of claim 10, wherein said trained time-varying treatment effect model comprises a G-formula approach.

15. The system of claim 10, wherein the processor is further configured to train the time-varying treatment effect model using historical patient data.

16. A non-transitory computer readable medium storing instructions that, when executed by one or more processors, cause the one or more processors to:
access a trained time-varying treatment effect model configured to predict a plurality of different outcomes for a patient using a plurality of patient outcome prediction features for the patient;
receive information about the patient, wherein the information comprises the plurality of patient outcome prediction features;
extract the plurality of patient outcome prediction features from the received information;
analyze, using the trained time-varying treatment effect model, the plurality of patient outcome prediction features to predict a plurality of different outcomes for the patient, wherein each of the plurality of different outcomes is associated with a patient treatment leading to said respective outcome;
identify at least one of the plurality of different outcomes and the treatment associated with the selected outcome as a recommended outcome and associated treatment for the patient, wherein identifying comprises identification of an outcome and associated treatment that maximizes favorable results for two or more organ systems for the patient; and
display a user interface configured to provide the recommended outcome and associated treatment for the patient to a user.

17. The non-transitory computer readable medium of claim 16, wherein the user interface is configured to provide two or more of the plurality of different outcomes treatments as possible or recommended outcomes and associated treatments for the patient.

18. The non-transitory computer readable medium of claim 17, wherein the instructions further cause the one or two processors to:
receive, via the user interface, a selection of one of the two or more of the plurality of different outcomes treatments provided via the user interface.

19. The non-transitory computer readable medium of claim 17, wherein identifying at least one of the plurality of different outcomes and the treatment associated with the selected outcome as a recommended outcome and associated treatment for the patient comprises the steps of:
comparing the predicted plurality of different outcomes to each other; and
selecting one of the plurality of different outcomes as an outcome that maximizes favorable results for two or more organ systems for the patient, if no other outcome has a more favorable outcome.

20. The non-transitory computer readable medium of claim 16, wherein the trained time-varying treatment effect model comprises a G-formula approach.
